SEQUENCE LISTING (2 sequences)

SEQ ID NO: 1 - DNA, 39 bp, Artificial
  CD40L Antagonist
ccgagatctcctataaatatgacctgcctcttccctaga

SEQ ID NO: 2 - DNA, 30 bp, Artificial
  CD40L Antagonist
cgggtcgaccctccaggatgactccccata

(19) United States
(12) Patent Application Publication
(10) Pub. No.: US 2006/0165688 A1
(43) Pub. Date: Jul. 27, 2006

(54) Remedies for pemphigus containing cd40l antogonist as the active ingredient

(76) Inventors: Masayuki Amagai, Tokyo (JP); Takeji Nishikawa, Tokyo (JP)

Correspondence Address: MORGAN & FINNEGAN, L.L.P., 3 WORLD FINANCIAL CENTER, NEW YORK, NY, 10281-2101, US

(21) Appl. No.: 10/510,001

(22) PCT Filed: Apr. 2, 2003
(86) PCT No.: PCT/JP03/04219

§ 371 Date: Sep. 30, 2004

(30) Foreign Application Priority Data
Apr. 3, 2002 (JP) 2002-101886

(51) Int. Cl. A61K 39/395; C07K 16/28

(52) U.S. Cl. 424/144.1; 530/388.22

(57) ABSTRACT

The present invention concerns developing a remedy for treating pemphigus by administering a CD40L antagonist to patients with pemphigus such as pemphigus vulgaris or pemphigus foliaceus, and a preventive agent for preventing the development of pemphigus by preventively administering a CD40L antagonist. Antagonists such as an anti-CD40L antibody that inhibit the interaction between a CD40L mediating the contact-dependent helper effector function on the T cell surface and a CD40 receptor on the antigen-presenting cell surface, were effective as a remedy and a preventive agent for pemphigus.

### TECHNICAL FIELD

The present invention relates to a remedy for treating pemphigus by administering a CD40L antagonist to patients who developed pemphigus, and a preventive agent to prevent the development of pemphigus by preventively administering a CD40L antagonist to patients who are likely to suffer from recurrence of pemphigus.

### BACKGROUND ART

It is elucidated that pemphigus is induced by autoantibodies against desmoglein (Dsg) which is a cadherin-type cell adhesion molecule found in desmosome (Amagai, M. et al., Cell, 67,869-877, 1991). There are Dsg1, Dsg2 and Dsg3 isotypes for Dsg: Dsg2 is expressed in all the tissues with desmosome including simple epithelium and heart, whereas expression of Dsg1 and Dsg3 are observed only in stratified squamous epithelium.

Pemphigus is classified into two major forms, pemphigus vulgaris (PV) and pemphigus foliaceus (PF). PV is further classified into the mucosal-dominant type in which mucosa is mainly attacked, and the mucocutaneous type in which the lesions are widely observed on not only mucosa but also skin. Patients with a mucosal-dominant PV are positive for anti-Dsg3 antibodies, and patients with PF are positive for anti-Dsg1 antibodies, and patients with mucocutaneous PF are positive for both anti-Dsg1 and anti-Dsg3 antibodies (Amagai et al., Jikken Igaku (Experimental Medicine) Vol. 19 No. 5 (extra number), 2001).

Pemphigus is treated with non-specific immunosuppressant agents, often with steroids, yet more specific immunosuppressant therapy has been required for pemphigus, clinical symptoms of which have been clarified to associate with antigens.

The object of the present invention is to identify a cancer-suppressing gene or a cancer gene useful for diagnosis and treatment of cancers such as human glioma, and to provide a method and an agent for diagnosing cancers such as human glioma, and to provide a method and an agent for treating cancers such as human glioma.

On the other hand, CD40, a cell surface molecule which has been cloned at a molecular level is identified on the surface of immature and mature B lymphocytes and is known to induce the proliferation of B cells when bound to antibodies (Eur. J. Immunol., 19, 1463-1467, 1989; J. Immunol., 140, 1425-1430, 1988; J. Immunol., 142, 4144-4152, 1989). In addition, CD40L, a ligand for CD40, has also been cloned at a molecular level (Nature, 357, 80-82, 1992; J. Exp. Med., 175, 1091-1101, 1992, EMBO J., 11, 4313-4319, 1992). It is also known that cells expressing the CD40L protein on their cell surface after having been transfected with the CD40L gene are capable of inducing proliferation of B cells as well as inducing production of antibodies along with other signal systems (Nature, 357, 80-82, 1992). In order to suppress humoral immunity by inhibiting the B-cell activation, it is also known to suppress humoral immunity by administering an antagonist, e.g. an anti-CD40L antibody, that inhibits the in vivo interaction between CD40L on T cells which activate B cells and CD40 on B cells (Japanese Patent Nos. 2840131, 3007977, 2974415, and 2991499). However, the fact that such immunosuppressant method is useful for pemphigus, an autoimmune disease, has been neither known nor verified.

The object of the present invention is to develop a remedy for treating pemphigus by administering a CD40L antagonist to patients who developed pemphigus, and a preventive agent for preventing the development of pemphigus by preventively administering a CD40L antagonist.

### DISCLOSURE OF THE INVENTION

The present inventors have attempted to generate model animals that develop pemphigus by inducing production of an antibody against Dsg3 which is an antigen for PV. It was, however, difficult to continuously produce an antibody against Dsg3 by immunizing wild-type mice because immunological tolerance to Dsg3 has already been established in those mice. Therefore, Dsg3^{-/-}, i.e. Dsg3 knockout mice, whose immunological tolerance to Dsg3 has not yet been established were immunized with a recombinant Dsg3, and splenocytes of the mice were then transferred to the immuno-deficient Rag2^{-/-} mice that had no mature T nor B cells (Amagai M. et al., J. Clin. Invest., 105, 625-631, 2000).

4 to 7 days after the transfer, production of IgG antibodies against Dsg3 was observed in the blood of mice that underwent the transfer, and the antibody production was sustained for as long as 6 months or longer. Deposition of anti-Dsg3 antibodies was confirmed on the cell membranes of stratified squamous epithelium such as skin, oral mucosa and esophagus of the mice, in addition to which, the suprabasilar acantholysis formation being a feature of pemphigus was observed on the oral mucosa and upper esophagus as a result of the fact that the cell-cell adhesion on the skin and mucosal epithelium had been impaired. The generated mice were concluded as being model mice with characteristic symptoms of pemphigus in view of clinical, pathological and immunological aspects. (It was also-confirmed that pemphigus was developed in the splenocytes of Dsg3^{-/-} mice without Dsg3 immunization.)

The present inventors have found out that production of the anti-Dsg3 antibody was entirely suppressed by preventively administering the anti-CD40L antibody, a CD40L antagonist, to the above-described model mice with pathologic conditions of pemphigus, and that the skin and mucosal lesions due to pemphigus can be prevented as a consequence. Further, administration of the anti-CD40L antibody after development of the disease yielded an inhibitory effect on production of the anti-Dsg3 antibody, and the phenotypes of some mice were shown to improve. The present invention, which involves using a CD40L antagonist as a remedy and a preventive agent for pemphigus, thus reached the completion.

An agent that inhibits the interaction between a CD40L receptor mediating the contact-dependent helper effector function on the surface of T cells and a CD40 receptor on the surface of an antigen-presenting cell, is capable of inducing immunological tolerance to the ongoing immune response in an antigen-specific manner, therefore such agent is anticipated to serve as a fundamental remedy for pemphigus which would replace non-specific immunosuppressant agents.

The present invention thus provides a remedy for pemphigus containing, as an active ingredient, an agent that inhibits the interaction between a CD40L receptor mediating the contact-dependent helper effector function on the T cell surface and a CD40 receptor on the antigen-presenting cell surface, and a preventive agent for pemphigus containing, as an active ingredient, an agent that inhibits the interaction between a CD40L receptor mediating the contact-dependent helper effector function on the T cell surface and a CD40 receptor on the antigen-presenting cell surface. An agent inhibiting the interaction is preferably an anti-CD40L antibody.

The present invention also provides a method for treating pemphigus wherein an agent that inhibits the interaction between a CD40L receptor mediating the contact-dependent helper effector function on the T cell surface and a CD40 receptor on the antigen-presenting cell surface, is administered to a patient with pemphigus, and a method for preventing pemphigus wherein an agent that inhibits the interaction between a CD40L receptor mediating the contact-dependent helper effector function on the T cell surface and a CD40 receptor on the antigen-presenting cell surface, is administered to a patient who is likely to develop pemphigus. An agent inhibiting the interaction is preferably an anti-CD40L antibody.

The present invention further provides the use of an agent that inhibits the interaction between a CD40L receptor mediating the contact-dependent helper effector function on the T cell surface and a CD40 receptor on the antigen-presenting cell surface, in the manufacture of a remedy for pemphigus. An agent inhibiting the interaction is preferably an anti-CD40L antibody.

### BRIEF DESCRIPTION OF DRAWINGS

FIG. 1 is a drawing showing the preventive effect on pemphigus by the administration of MR1 antibody.

FIG. 2 is a drawing showing the therapeutic effect on pemphigus by the administration of MR1 antibody.

### BEST MODE OF CARRYING OUT THE INVENTION

Various aspects of the present invention will be explained in detail with regard to the following items.
1. CD40L antagonist: A CD40L antagonist is defined as an agent that inhibits the interaction between a CD40L receptor mediating the contact-dependent helper effector function on the surface of T cells and a CD40 receptor on the surface of an antigen presenting cell. CD40L antagonists include not only agents that interact with CD40L but also agents that interact with CD40. CD40L antagonists may be antibodies against CD40L (e.g. monoclonal antibodies against CD40L), antibody fragments to CD40L (e.g. Fab or (Fab′)₂ fragments), chimeric or humanized antibodies, soluble CD40 or soluble CD40L or fragments thereof, or other compounds inhibiting the interaction between CD40L and CD40.

A property of a CD40L antagonist which is to inhibit the interaction between CD40L and CD40 can be determined from whether the binding of a labeled soluble CD40 to an activated helper T cell is suppressed or not. A labeled soluble CD40 can be prepared by producing a soluble CD40 according to, for instance, the method of Example 1 in Japanese Laid-Open Patent Application No. 6-220096, and subsequently by labeling the CD40 with an appropriate labeling substance such as a fluorescent substance and radioactive isotope. The binding of a labeled soluble CD40 to the activated helper T cells can be assessed by FACS using, for example, a fluorescent-labeled soluble CD40.
2. Anti-CD40L antibody: Mammals (e.g. mice, hamsters or rabbits) can be immunized with the CD40L protein or a protein fragment (e.g. a peptide fragment) which is in the form of an immunogen causing immune response in the mammals.

The CD40L protein can be expressed and purified as follows: An expression vector incorporating the CD40L cDNA (Armitage et al., Nature, 357, 80-82, 1992, Hollembaugh et al., EMBO J., 11, 4313-4319, 1992) is expressed in a host cell such as bacteria or mammal cell lines and the CD40L protein is purified from a culture solution according to a standard protocol. Also, it may be expressed as a fusion protein with, for example, GST and the like. When expressed as a fusion protein with GST, the CD40L protein may be purified using a glutathione column. A CD40L peptide can be synthesized by a known method (e.g. F-moc or T-boc chemical synthesis) based on the amino acid sequence of CD40L (Armitage et al., Nature, 357, 80-82, 1992; Hollembaugh et al., EMBO J., 11, 4313-4319, 1992), and immunogenicity of the synthesized peptide may be enhanced through binding with an appropriate carrier, for example, KLH.

Anti-serum can be obtained after immunization of thus purified CD40L protein or a peptide fragment with an adjuvant, and a polyclonal antibody can be isolated from the anti-serum if so desired. Further, for producing a monoclonal antibody, antibody-producing cells (lymphocytes) are collected from the immunized animals, which cells are then immortalized by fusing with myeloma cells according to a standard cell-fusion method to obtain hybridoma cells. This technique is a method that has been established in the art and can be carried out according to an appropriate manual (Harlow et al, Antibodies: A Laboratory Mannual, 1998, Cold Spring Harbor Laboratory). Still further, a monoclonal antibody may be generated by other methods for producing human monoclonal antibodies including human B-cell hybridoma technique (Kozbar et al., Immunol. Today, 4, 72, 1983), EBV-hybridoma method (Cole et al., Monoclonal Antibody in Cancer Therapy, 1985, Allen R. Bliss, Inc., pages 77-96), and screening of a combinatorial antibody library (Huse et al., Science, 246, 1275, 1989).

In the present description, antibodies include antibody fragments that specifically bind to CD40L, for example, Fab or (Fab′)₂ fragments.

Mouse monoclonal antibodies generated from animals other than humans, e.g. mice, often raise immune response to these monoclonal antibodies, since they are recognized as being foreign proteins when administered to humans. One of the means to avoid such problem to take place is a chimeric antibody, i.e. an antibody whose antigen-binding region is derived from a mouse monoclonal antibody and the other regions from a human antibody. In the present invention, antibodies include chimeric antibodies as well. Such chimeric antibodies include a chimeric antibody using the whole variable region of a mouse monoclonal antibody as an antigen-binding region (Morrison et al., Proc. Natl. Acad. Sci. USA, 81, 6851, 1985, Takeda et al., Nature, 314, 452, 1985), and a chimeric antibody using a combination of a framework region from human and a super-variable region from a mouse-monoclonal antibody as an antigen-binding region (Teng et al., Proc. Natl. Acad. Sci. USA, 80, 7308-12, 1983, Kozbar et al., Immunol. Today, 4, 7279, 1983), yet the present invention will not be limited to these exemplifications.

A remedy for pemphigus of the present invention may be administered to patients with pemphigus. Further, a preventive agent for pemphigus of the present invention may be administered to patients who are likely to develop pemphigus for the purpose of preventing the development of pemphigus.

Administration of a remedy and a preventive agent for pemphigus of the present invention may be carried out by usual protocols such as injection (subcutaneously, intravenously, etc.).

Formulation of a remedy and a preventive agent for pemphigus is appropriately selected based on the means of administration. For example, pharmaceutical compositions suitable for the injection use include a sterilized aqueous solution (if water-soluble) or dispersion solution, and a sterilized injection solution or a sterilized powder for instantly preparing a dispersion solution. Pharmaceutical compositions suitable for the injection use should always be sterilized and they should be fluid enough to the extent that an injector can be handled easily. The compositions should be stable under the manufacture and storage conditions and should be protected from the action of contaminated microorganisms such as bacteria and fungi. Carriers may be, for example, water, ethanol, polyol (e.g. glycerol, propylene glycol, polyethylene glycol, etc.) and vehicles comprising suitable mixtures of these or may be dispersion vehicles. A suitable fluid property can be maintained by applying coating with such as lecithin, and for a dispersion solution, by maintaining a necessary particle size and by using surfactants. Protection from the action of bacteria is actualized with various antibacterial agents and antifungal agents, such as paraben, chlorobutanol, phenol, ascorbic acid and thimerosal. In many cases, it would be suitable that the compositions contain isotonic agents including sugar, polyalcohol such as mannitol and sorbitol, and sodium chloride. The injection compositions can be absorbed sustainedly by compounding agents for delaying absorption, such as aluminum monostearate or gelatin, in the compositions.

An injection solution can be prepared by compounding a requirement of an active compound (such as a CD40L antagonist) in a suitable vehicle along with the above-mentioned ingredients if necessary either alone or in combinations, and then by filtering for sterilization. Preparation of a dispersion solution is generally carried out by compounding an active compound in a sterilized vehicle which contains a basic dispersion vehicle and other necessary ingredients selected from those shown above. Sterilized powder for preparing a sterilized injection solution is preferably prepared by vacuum- and freeze-drying methods, by which powder comprising an active ingredient and the desired additional ingredients having been filtered for sterilization in advance can be obtained.

Dose of a remedy for pemphigus is an amount sufficient for treating pemphigus, and could vary depending on age, sex, drug-sensitivity and the disease history of a patient, administration methods and soon. Moreover, dose of a preventive agent for pemphigus is an amount sufficient for treating pemphigus, and could vary depending on age, sex, drug-sensitivity and the disease history of a patient, administration methods and so on.

The present invention will be described in more detail with the reference to the following Examples that will not limit the scope of the present invention.

MR1, an anti-CD40L antibody (Noelle et al., Proc. Natl. Acad. Sci. USA, 89, 6550-54, 1992), as used in the following Examples is a monoclonal antibody against a mouse CD40L generated with the use of hamsters, and is available from PharMingen (Catalog No.: PM-09020D or PM-09021D). MR1-producing cells are available from American Type Culture Collection (ATCC) (ATCC No.: HB-11048).

Dsg3^{-/-} mice used for the Examples are available from The Jackson Laboratory, Bar Harbor, Me., USA and Rag2^{-/-} mice from Tacnin Farms, Germantown, N.Y., USA.

In the Examples, a recombinant Dsg3 (rDsg3) was generated by amplifying the extracellular domain of mouse Dsg3 (GenBank U86016) by PCR using primers comprising the sequences shown by SEQ ID Nos. 1 and 2, and following the method described in Amagai M., et al., J. Clin. Invest., 94, 59-67, 1994. The antibody titer was determined by ELISA method described in Amagai M., et al., J. Clin. Invest., 105, 625-631, 2000.

### EXAMPLE 1

### Generation of Model Mice with Pathologic Conditions of Pemphigus

Model mice with pathologic conditions of pemphigus were generated according to the method of Amagai et al (Amagai M., et al., J. Clin. Invest., 105, 625-631, 2000).

The rDsg3-immunized Dsg3^{-/-} mice were killed under ether anesthesia and the spleens were extracted. The splenocytes were prepared under sterile condition and then suspended in PBS at 1×10⁸/ml, and subsequently 0.5 ml of the suspension (5×10⁷ cells) was intravenously injected to Rag2^{-/-} mice via the tail veins.

Production of IgG antibodies against Dsg3 was observed in the blood of recipient mice 4 to 7 days after the transfer and this antibody production was sustained for as long as 6 months or longer. The Dsg3 antibody deposition was confirmed on the cell surface of stratified squamous epithelia such as skin, oral mucosa and esophagus of the mice, in addition to which, the suprabasilar acantholysis, characteristic feature of pemphigus vulgaris, was observed on the oral mucosa and upper esophagus as a result of the impaired cell-cell adhesion on the skin and mucosal epithelia.

### EXAMPLE 2

### The Preventive Effect on Pemphigus by the Administration of MR1 Antibody

It was examined whether the transferred splenocytes were capable of inducing immunological tolerance to Dsg3 when MR1 antibody was preventively administered so that CD40L was present at the time of inducing immune response to Dsg3.

500 μg each of MR1 antibody and the control hamster IgG was intraperitoneally administered to recipient mice (Rag2^{-/-} mice) (n=5) 2 days before the transfer of Dsg3^{-/-} mouse splenocytes and 0, 2, 4 and 7 days after the transfer.

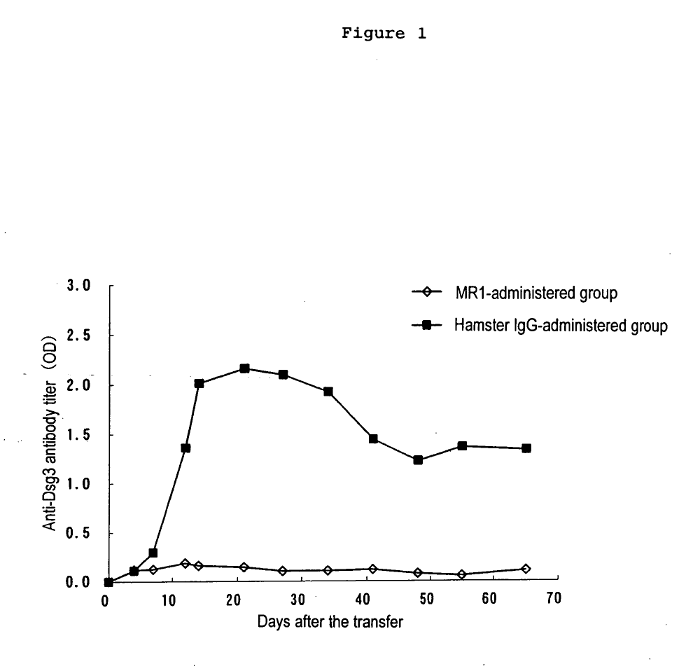

Production of the anti-Dsg3 antibody was confirmed in the control group 14 days after the transfer, while any apparent antibody production or phenotype was not observed at all throughout the observation period of 66 days in the MR1-administered mice (FIG. 1). In addition, weight loss, hair loss in resting period and the immediate suprabasal acantholysis which is a pathohistological feature of PV was observed in the control group, whereas neither weight loss nor symptoms of PV was observed in the MR1 administered group. The MR1 antibody apparently showed a preventive effect on PV.

### EXAMPLE 3

### Therapeutic Effect on Pemphigus by the Administration of MR1 Antibody

MR-1 antibody was administered to the mice that had been introduced with the Dsg3^{-/-} mouse splenocytes and that had exhibited development of PV as shown by their production of antibodies against Dsg3, and the presence or absence of any therapeutic effect was examined.

Recipient mice (Rag2^{-/-} mice) introduced with the non-immunized Dsg3^{-/-} mouse splenocytes were administered 1 mg each of MR1 and hamster IgG twice a week for 6 weeks, for the total of 12 times, starting from 7 weeks after the transfer of splenocytes (n=10). Untreated PV model mice were monitored at the same time to see a usual change in antibody titers (n=5).

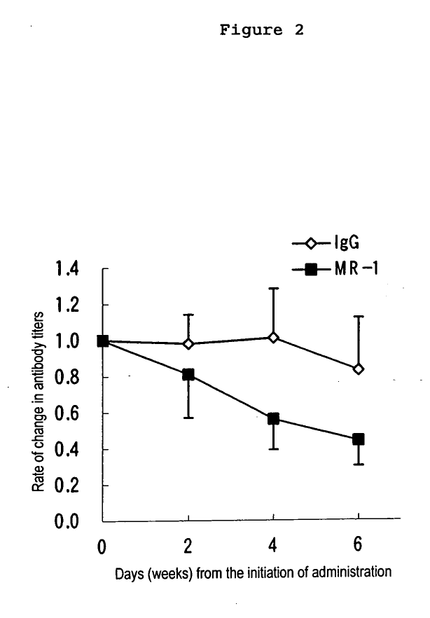

Blood was collected 0, 2, 4 and 6 weeks after the initiation of administration and antibody titers to Dsg3 was measured. Statistical analysis was carried out except for mice that showed high antibody titers (mice showing 2000 or higher fold-increase in antibody titers) before the administration and died during the administration period, and also except for mice whose antibody titers before the administration were so low (mice showing 100 or lower fold-increase in antibody titers) that no therapeutic effect could be anticipated. As shown in FIG. 2, antibody titers decreased to 30% of the level of before the administration in the MR1 antibody administered group, whereas no decrease in antibody titers was observed in the control group. During the administration period, some mice showed amelioration in PV, however, no statistically significant difference in the PV symptoms was observed in both groups.

### INDUSTRIAL APPLICABILITY

As a result of administration of g39 antagonist, it was confirmed that production of the anti-Dsg antibody was suppressed as well as the skin and mucosal lesions due to pemphigus were ameliorated. Therefore, it was shown that a CD40L antagonist could serve as a fundamental remedy and a preventive agent for pemphigus.

1. A remedy for pemphigus containing, as an active ingredient, an antagonist that inhibits the interaction between a CD40L receptor mediating the contact-dependent helper effector function on the T cell surface and a CD40 receptor on the antigen-presenting cell surface.

2. The remedy for pemphigus according to claim 1, wherein the antagonist that inhibits the interaction is an anti-CD40L antibody.

3. A preventive agent for pemphigus containing, as an active ingredient, an antagonist that inhibits the interaction between a CD40L receptor mediating the contact-dependent helper effector function on the T cell surface and a CD40 receptor on the antigen-presenting cell surface.

4. The preventive agent for pemphigus according to claim 3, wherein the antagonist that inhibits the interaction is an anti-CD40L antibody.
